(19) United States
(12) Patent Application Publication
(10) Pub. No.: US 2009/0260626 A1
(43) Pub. Date: Oct. 22, 2009

(54) Inhaler for Powdery, Especially Medical Substances

(76) Inventor: Alfred Von Schuckmann, Kevelaer (DE)

Correspondence Address: COLLARD & ROE, P.C., 1077 NORTHERN BOULEVARD, ROSLYN, NY, 11576, US

(21) Appl. No.: 11/988,858

(22) PCT Filed: Jul. 4, 2006
(86) PCT No.: PCT/EP2006/063851

§ 371 Date: Jan. 15, 2008

(30) Foreign Application Priority Data
Jul. 18, 2005 (DE) 10 23005 033 397.

(51) Int. Cl. A61M 15/00

(52) U.S. Cl. 128/203.15

(57) ABSTRACT

The invention relates to an inhaler (1) for powdery, especially medical substances (17), comprising an air duct (45) that leads to a mouthpiece (3), a reservoir (16) for the substance (17), and a moved dosing chamber (D) for apportioning a specific amount of substance from the reservoir (16) into the zone of a transfer point (U) to the air flow. A filling means (B) which charges the dosing chamber (D) similar to a shovel is moved towards the dosing chamber (D) so as to penetrate the substance supply. In order to create a simple design for such an inhaler in such a way that the same is easy and functionally safe to handle regarding apportioning a reproducible amount of substance, the shovel-type charging process performed by the filling means (B) is derived from a relative movement between the dosing chamber (D) and the reservoir (16).

The invention relates in first instance to an inhaler for powdery, especially medical substances, with an air channel leading to a mouthpiece, a storage chamber for the substance and a moved dosing chamber for apportioning a specific amount of substance from the storage chamber into the region of a transfer point to the air stream, a filling means that charges the dosing chamber in a scoop-like manner being moved right through the supply of substance to the dosing chamber.

An inhaler of the type in question is known for example from DE 101 06 788 A1. There, the dosing chamber is formed by a perforated plate, the apportioning of a reproducible amount of substance being achieved by relative displacement of a scooping part with respect to the filling means, which extends right through the supply of substance. Said filling means takes the form of a deflecting wall which can be overrun. The displacement for apportioning the specific amount of substance is derived from a turning movement to be carried out manually.

With regard to the prior art described above, one technical problem of the invention is seen as being that of configuring an inhaler of the type in question in a structurally simple way, so as to obtain simple handling that is functionally reliable in terms of apportioning a reproducible amount of substance.

This problem is solved first and foremost by the subject matter of Claim 1, it being provided that the scoop-like charging by the filling means that moves right through the supply of substance is derived from a relative movement between the dosing chamber and the storage chamber. As a result of such a configuration, a structurally simple, functionally reliable inhaler is achieved. The scoop-like charging by the filling means takes place for example just by deriving a relative movement for the displacement of the dosing chamber from a filling position into the transfer position, which relative movement may also take place in a positively controlled manner, thus furthermore for example when the inhaler is put into operation. The relative displacement of the dosing chamber with respect to the storage chamber preferably takes place here in a straight line along a longitudinal axis of the inhaler. The scooping movement of the filling means on the other hand preferably takes place offset at an angle to that.

The subjects of the further claims are explained below with respect to the subject matter of Claim 1, but may also be of importance in their independent formulation.

Thus it is provided in a development of the subject matter of the invention that the relative movements of the dosing chamber on the one hand and of the filling means on the other hand take place substantially in directions perpendicular to one another relative to the storage chamber. Thus displacement of the dosing chamber along a central, possibly vertical, inhaler axis is preferred, while the filling means is movable about a (horizontal) axis extending transversely thereto. Furthermore, the filling means is articulated in such a way that, when there is a relative movement between the dosing chamber and the storage chamber, it effects a charging of the dosing chamber in a scoop-like, even loosening, manner, moving through the supply of substance, by a preferably radial displacement, and as a result with a constantly reproducible amount of substance. The dosing chamber is preferably formed in a rod-like body extending through the storage chamber, furthermore preferably extending centrally through it. For apportioning the amount of substance, both the dosing chamber and the associated filling means are disposed within the storage chamber, at the same time a constant loosening of the stored substance in the storage chamber being achieved as a result of the relative movement of the individual parts in relation to one another. The rod-like body at the same time forms a conduit portion. Said body leads to the inhaler mouthpiece for possible clearance of the storage chamber by means of suction air acting upon it in the course of inhalation. The dosing chamber is hermetically separated thereby from the conduit portion. This ensures that the dosing chamber cannot be cleared by suction air before complete and exact filling. This is achieved for example by sealing off the conduit portion on one side from the surroundings until the inhaler is ready to use. The scoop-like filling means is connected to a body forming the dosing chamber, for example to the rod-like body, which at the same time forms the conduit portion. In a corresponding way, the filling means is at least initially firmly connected to the body forming the dosing chamber, as a result of which the filling means is moved along together with the dosing chamber, thus furthermore in particular along the central axis of the inhaler, when there is a relative movement between the dosing chamber and the storage chamber. The movement of the filling means toward the dosing chamber takes place as a result of a sloping positioning of the storage chamber wall with respect to the direction of movement of the dosing chamber, which sloping storage chamber wall effects a positive control on the filling means in the direction of the dosing chamber. As a result of this, the scoop-like charging of the dosing chamber with the substance takes place, the reproducible amount of said substance depending on the size of the dosing chamber. The positive control of the filling means preferably takes place from radially outside to radially inside in the direction of the dosing chamber, preferably formed on the rod-like body. In a preferred configuration of the subject matter of the invention, the dosing chamber is formed as an annular groove on the outer side of the wall of the rod-like body. The annular groove preferably extends here in a plane which is transverse in relation to the extent of the rod. Four filling means are preferably provided, together being connected to the body forming the dosing chamber and, as a result of the sloping position of the storage chamber wall, being positively displaced in the direction of the portions of the dosing chamber. These filling means are spaced apart at equal angles in relation to one another about the axis of the rod-like body and in the filling position form a closed ring, at least in the region directly associated with the annular groove of the dosing chamber. The substance forced in the direction of the dosing chamber correspondingly cannot deviate from the intended path, making it possible for an exact, reproducible amount of substance to be apportioned. More preferably, the filling means are produced from an elastic plastic or the like, so that, once the positively controlled inward displacement in the direction of the dosing chamber has been discontinued, discontinuance of the action exerted by the sloping walls results in the springing-back movement of the filling means into their starting position. This may be initiated for example by the filling means being connected in a latching manner to the dosing chamber body in such a way that the latch can be overcome. In the course of the relative movement between the dosing chamber and the storage chamber and the accompanying action exerted on the filling means to make them pivot radially inward, the relative movement between the dosing chamber and the storage chamber is continued, for the displacement of the dosing chamber into the transfer point to the air stream. The filling means thereby overruns the latching connecting region to the body of the dosing chamber, for example the rod-like body; as a result of the spring force then released, the pincer-arm-like filling means moves back again into the starting position. Following inhalation and the corresponding concluding displacement of the dosing chamber and the storage chamber back into the starting position, the filling means is again captured in a latching manner by the body of the dosing chamber.

The invention also relates to an inhaler for powdery, especially medical substances, with an air channel leading to a mouthpiece, a storage chamber for the substance and an at least linearly moved dosing chamber for apportioning a specific amount of substance from the storage chamber and bringing the amount of substance into a transfer position, for transfer to the air stream, it being possible furthermore by relieving a compression spring to produce a stream of compressed air for blowing the substance out of the dosing chamber independently of aspiration.

An inhaler of the type in question is known for example from DE 40 27 391 A1. The amount of substance to be discharged in the dosing chamber is brought into the transfer position by linear displacement of said chamber by means of a pushbutton, a degree of displacement for an air plunger being enabled at the same time in the course of this linear displacement of the dosing chamber, which air plunger acts upon an air storing space. By releasing the spring force of a compression spring acting upon the air plunger, a stream of compressed air is produced, blowing the apportioned substance out of the dosing chamber in the direction of the air channel.

With regard to the prior art described above, one technical problem of the invention is seen as being that of configuring an inhaler of the type in question in a structurally simple way, simple in terms of handling and functionally reliable.

This problem is solved first and foremost by the subject matter of Claim 11, it being provided that the inhaler can be closed by a closure cap and that the compression spring is tensioned as the closure cap is placed onto the inhaler. As a result of this configuration, an inhaler which is simple in terms of handling and functionally reliable is provided. In addition, a structurally simple solution is achieved as a result. The inhaler has a protective cap, which covers over the mouthpiece leading to the mouth in a protective manner when the inhaler is not in use. Correspondingly, the protective cap has to be removed to allow the inhaler to be used. The relative movement between the closure cap and the inhaler is used for tensioning the compression spring. Placing the closure cap onto the inhaler after inhalation automatically brings about the tensioning of the compression spring for the next time it is used. If in this respect a screw closure cap is used, force transmission that is simple in terms of handling can be achieved as a result for the tensioning of the compression spring. Since inhalers of the type in question are also used in emergency cases, the measure of tensioning the compression spring already in advance after the last time it is used proves to be a major advantage with regard to handling.

The subjects of the further claims are explained below with reference to the subject matter of Claim 11, but may also be of importance in their independent formulation. Thus it is further proposed that the dosing chamber is moved into the filling starting position as the closure cap is placed onto the inhaler. The dosing chamber correspondingly preferably lies in the storage space, filled with the powdery substance to be inhaled, when the inhaler is in the position in which it is not in use. It lies furthermore in a position in which an air flow path to the surroundings outside is closed. As a result of this, in the not-in-use position, the dosing chamber is at the same time protected from the ingress of atmospheric moisture. This counteracts the formation of lumps in the amount of substance to be introduced into the dosing chamber. The dosing chamber is preferably moved toward an air storing space, which can be acted upon by means of the compression spring, as the closure cap is removed. As a result of this configuration, substantially all that is needed for handling the inhaler is to operate the closure cap, that is initially by screwing the same onto the inhaler, to tension the compression spring, and by unscrewing the closure cap as the inhaler is put into operation, to displace the apportioned amount of substance into the flow path or in the direction of the air storing space that can be acted upon by the compression spring. As the inhaler is put into operation, removal of the closure cap at the same time allows filling of the dosing chamber to take place by means of a filling means acting in a scoop-like manner. Very simple handling of the inhaler is obtained. Immediately after removal of the closure cap from the inhaler, the latter is already immediately ready for inhalation of the apportioned amount of substance. No further, possibly awkward, handling of the inhaler is needed. The air storing space forms between an air plunger, acted upon by the compression spring, and a plunger receiving wall as the closure cap is removed. Furthermore, the air storing space is in flow communication with the air channel leading to the mouthpiece, it being possible furthermore for the dosing chamber that is filled with an amount of substance to be brought into the path between the air storing space and the mouthpiece. Thus, preferably, the dosing chamber is disposed such that it radially coincides with the air storing space before the compression spring is triggered, the dosing chamber communicating furthermore with a conduit portion, through which the air/substance mixture can be discharged in the direction of the mouthpiece, the compressed air being able to flow from the air storing space into the conduit, clearing the dosing chamber, through a radial opening disposed at a distance from the dosing chamber, as a result of which the substance is forced in a specific manner out of the dosing chamber by means of the compressed air. The clearing of the dosing chamber is further assisted by the compressed air flowing out of the air storing space to the dosing chamber in tangential alignment. In the case of a proposed configuration of the dosing chamber as an annular groove, a kind of circular flow is achieved in this way. As a result, complete emptying of the dosing chamber is achieved, which is a condition for the exact dosing of the medicament to be transferred. The dosing chamber and the conduit are preferably disposed in a central rod-like body that is fixed relative to the inhaler housing. This is more preferably centrally aligned coaxially in relation to the inhaler housing. The release of the compression spring acting upon the air plunger can take place by actuating a triggering button. In the ready position after removal of the closure cap, the compression spring or the air plunger is held in a latched manner in the tensioned position. By actuating the triggering button, this latching is discontinued. Further simplification of the handling is achieved by the triggering of the compression spring being performed by the user himself, as a result of producing a suction pressure. Thus the triggering can be achieved in an extremely simple way by breathing in strongly, as a result of which it is also ensured that the user develops the required rate of flow when breathing in.

The invention is explained in more detail below with reference to the accompanying drawing, which merely represents an exemplary embodiment and in which:

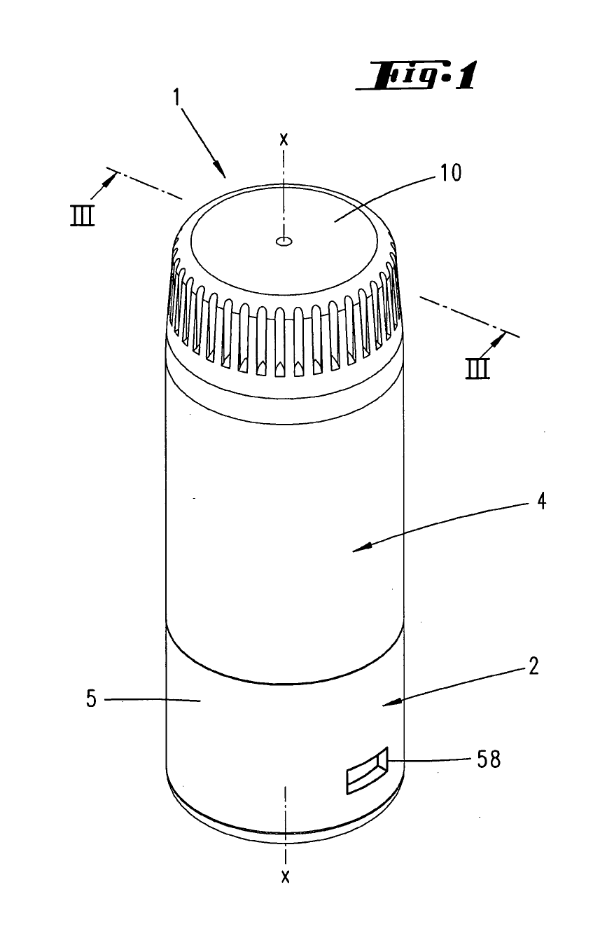

FIG. 1 shows the inhaler with the closure cap placed on, in a perspective representation;

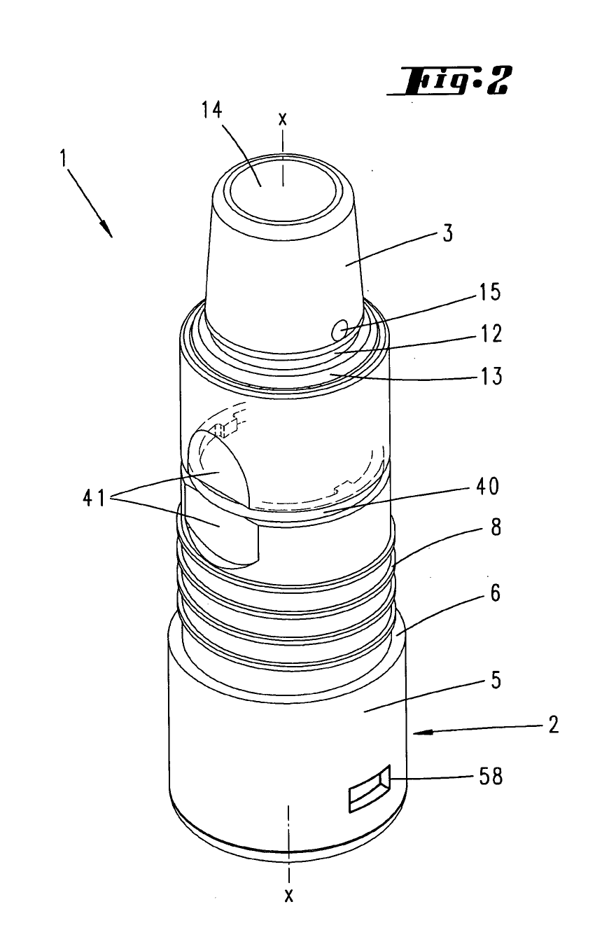

FIG. 2 shows the inhaler in the ready position after removal of the closure cap;

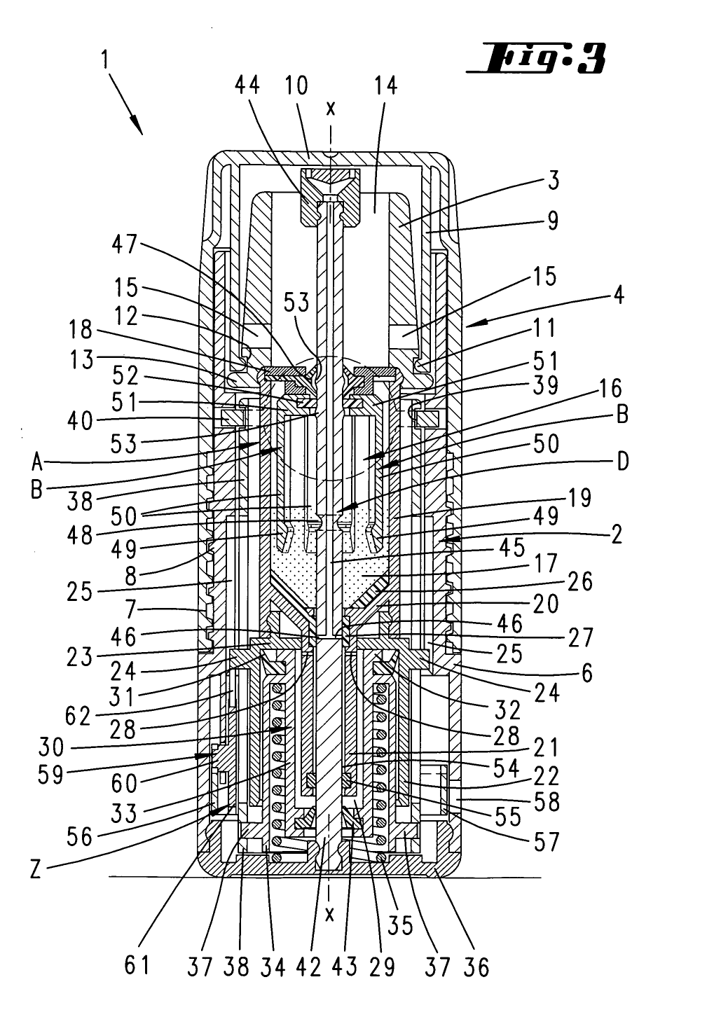

FIG. 3 shows the longitudinal section along the line III-III in FIG. 1, for the closed initial state of the inhaler;

FIG. 4 shows a sectional representation corresponding to FIG. 3, but for an intermediate position as the closure cap is removed, in which intermediate position charging of the dosing chamber takes place by means of a filling means;

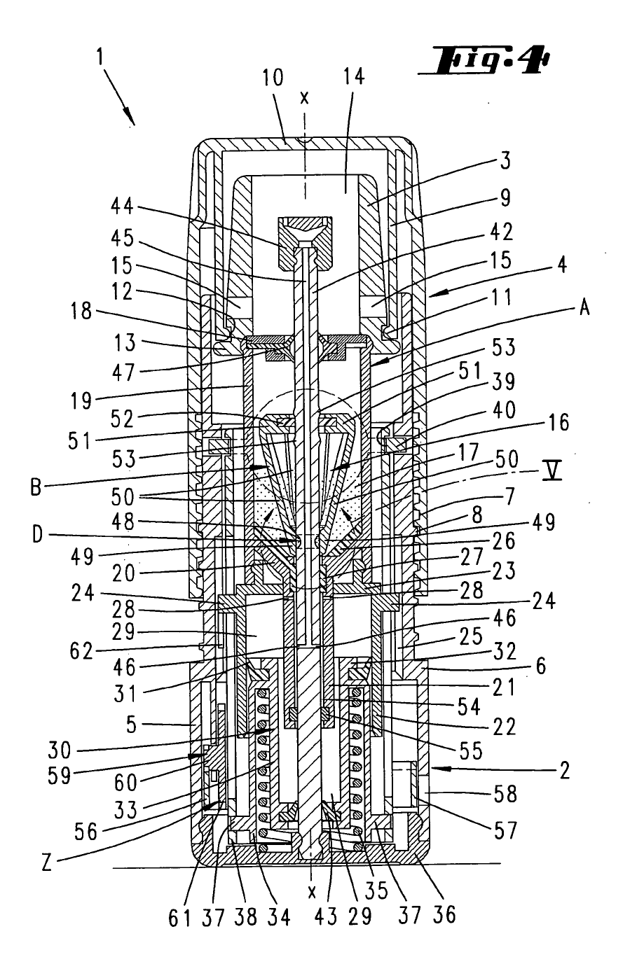

FIG. 5 shows the enlargement of the region V in FIG. 4;

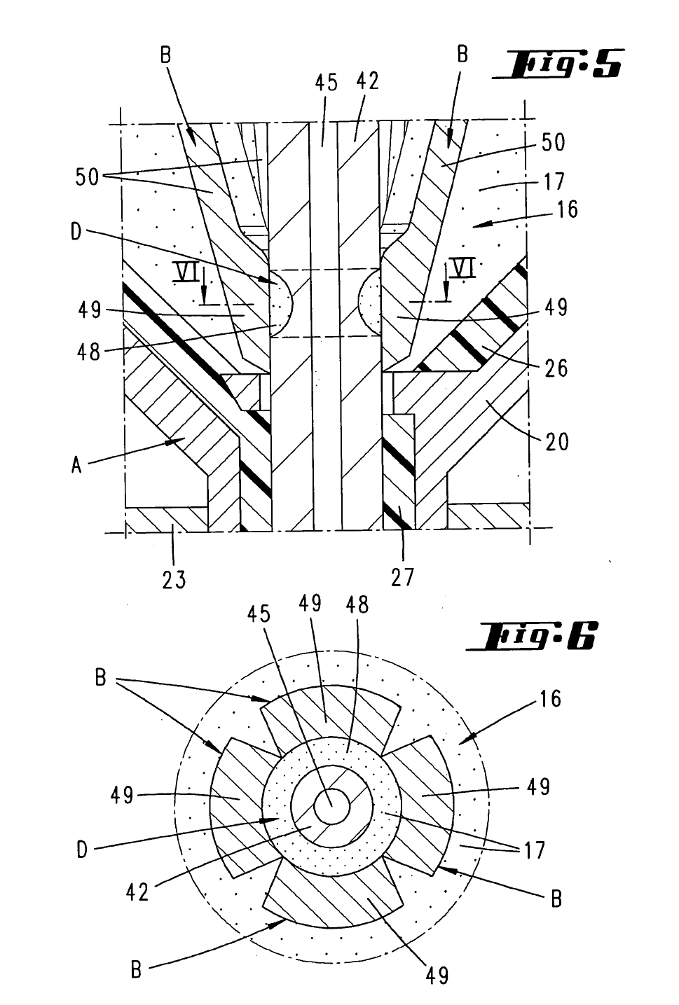

FIG. 6 shows the section along the line VI-VI in FIG. 5;

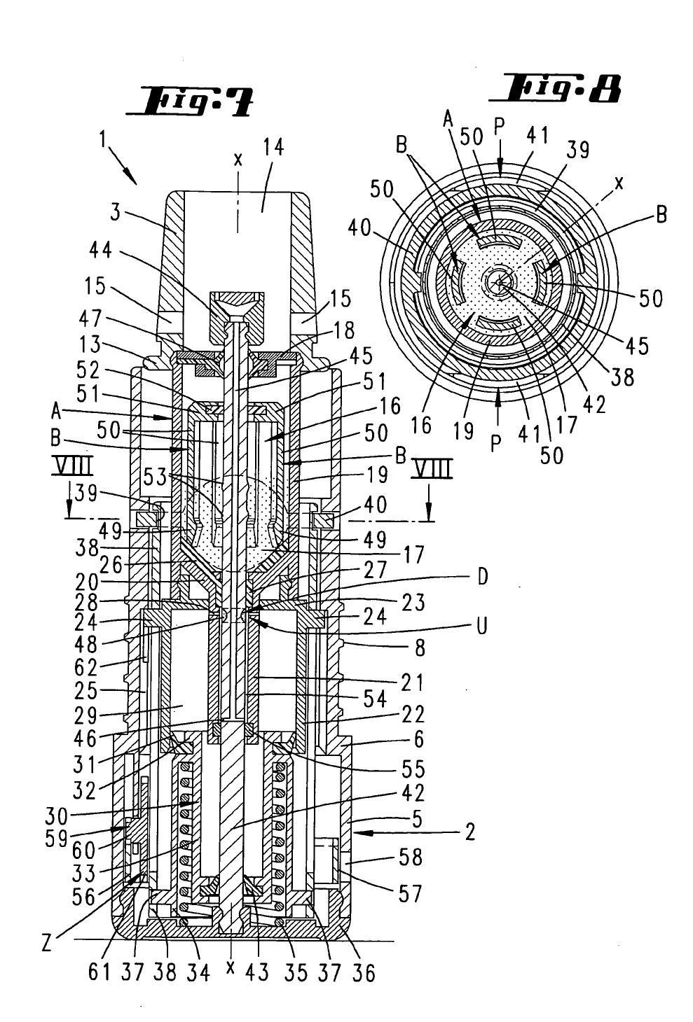

FIG. 7 shows a further sectional representation according to the representation in FIG. 3, but after removal of the closure cap and the ready position of the inhaler thereby achieved;

FIG. 8 shows the cross section along line VIII-VIII in FIG. 7;

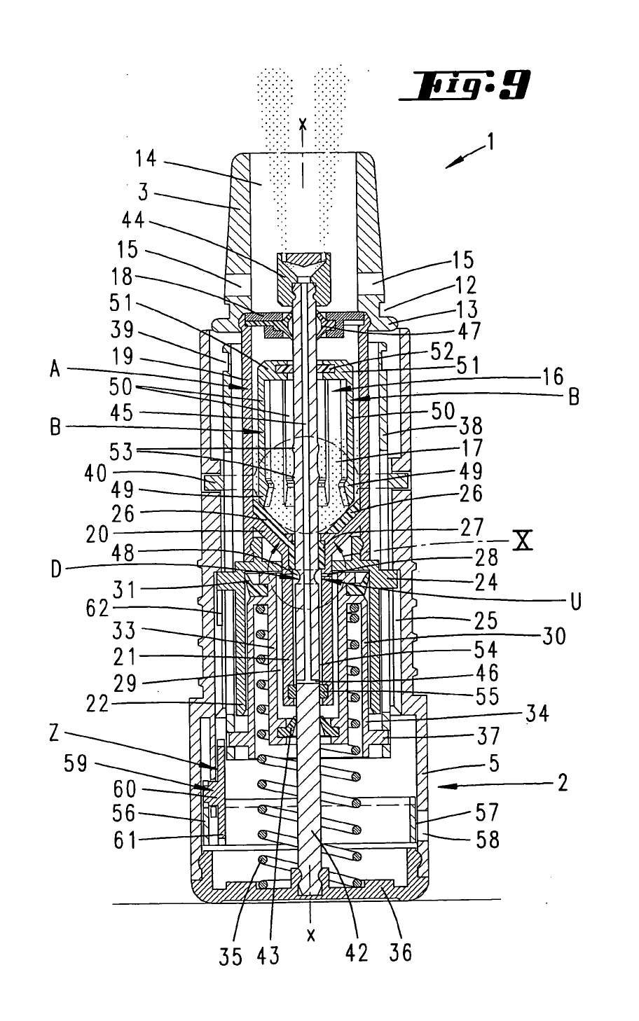

FIG. 9 shows a sectional representation corresponding to FIG. 7, but after triggering of a compression spring acting on an air storing space and the accompanying blowing out of the apportioned substance through the mouthpiece;

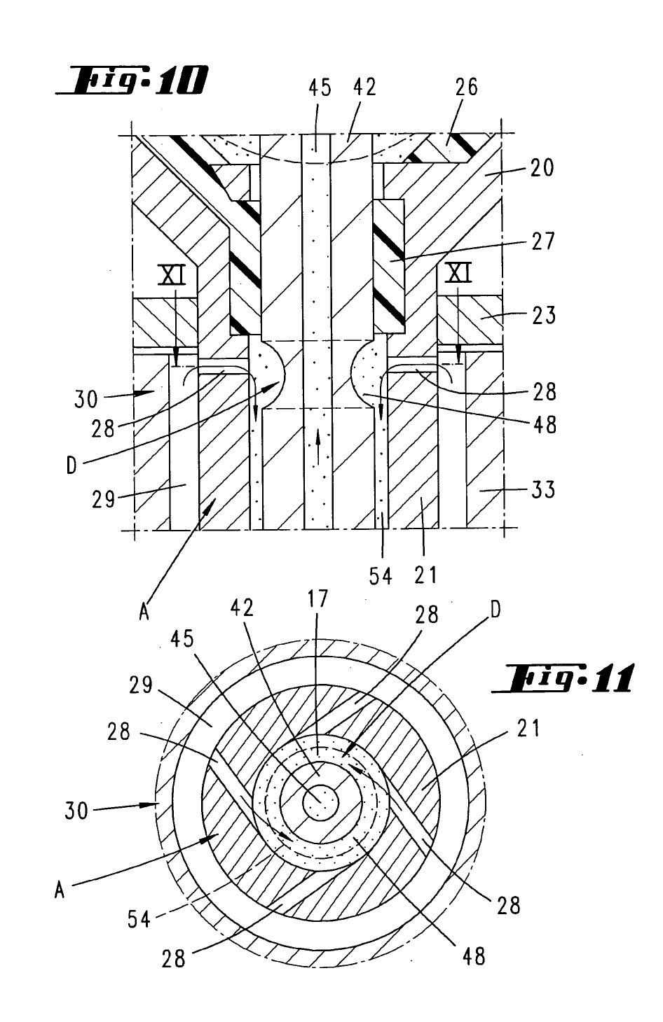

FIG. 10 shows the enlargement of the region X in FIG. 9;

FIG. 11 shows the section along the line XI-XI in FIG. 10.

The inhaler 1 represented is given the form of a pocket device that can be conveniently carried. The form is determined here by a cylindrical housing 2. Altogether, the inhaler 1 has a rotationally symmetrical construction.

The housing 2, which hollow-cylindrical as a whole, receives in the region of an end that is open on the user side a mouthpiece 3, which can be displaced in the axial direction of the inhaler 1. This mouthpiece is flattened appropriately for a mouth and, in a not-in-use position of the inhaler 1 according to the representation in FIG. 3, stays in a lowered position, in which the mouthpiece 3 protrudes only partially above the facing periphery of the housing of the inhaler 1.

A further component part of the inhaler 1 is a closure cap 4. This likewise has a cylindrical shape, with an outside diameter that corresponds to the maximum outside diameter of the inhaler housing 2. The closure cap 4 engages over the housing 2 in the region of a region of the housing that is reduced in diameter to match. In approximately the lower third of the housing 2, a step-like radial offset of the housing wall 5 toward the outside is provided. The housing step 5 formed as a result forms a stop face for the closure cap 4.

The closure cap 4 is screw-connected to the inhaler housing 2, for which purpose the closure cap 4 has an internal thread 7 on the inner wall and, associated with it, the housing wall 5 has an external thread 8. This is a steeply sloping thread.

The closure cap 4 serves not only for closing the inhaler housing 2 and protecting the mouthpiece 3 but also additionally for preparing the inhaler 1 for the discharge of substance that is to take place. For this purpose, the closure cap 4 has on the inside a driver 9, which is in the form of an annular wall, is coaxial in relation to the overall axis x-x and is connected to the underside of the top 10 of the closure cap protruding above the mouthpiece 3. This driver 9 has in the region of the free end that is remote from the top 10 a radially inwardly protruding driving bead 11. In the not-ready position of the inhaler 1, this engages in an annular groove 12 at the base of the mouthpiece 3. On the underside of this annular groove 12, the mouthpiece wall goes over into a radially widening collar, which at the same time forms a supporting face for the driver 9 on the closure cap.

The mouthpiece 3 forms in the usual way a central discharge opening 14. At the bottom of this central discharge opening 14, radially aligned air inlets 15 are formed in the mouthpiece wall. In a position for use, these are open toward the surroundings.

Held at the bottom of the mouthpiece 3 is a storage chamber 16 for the powdery, especially medical substance 17. The storage chamber 16 is formed as a rotationally symmetrical component and is secured with positive engagement to the bottom of the mouthpiece 3 in the region of an upper peripheral edge facing the mouthpiece 3.

A chamber top 18, aligned perpendicular to the axis of rotation x, forms the upper termination of the storage chamber 16 and at the same time the bottom termination of the discharge opening 14 of the mouthpiece.

In the region remote from the chamber top 18, the storage chamber wall 19 goes over into a base portion 20, falling away radially inward in a sloping manner. This is as a whole formed in the shape of a funnel, with an angle of inclination of approximately 45°.

This chamber base 20 goes over into a centrally suspended, tubular transfer portion 21, molded on integrally in the same material. This portion extends coaxially in relation to the axis of rotation x with an inside diameter which corresponds approximately to one quarter of the inside diameter of the storage chamber 16.

The transfer portion 21 is radially enclosed by a pot-shaped, coaxially aligned guiding portion 22. This extends approximately over the entire axial length of the transfer portion 21 and in the bottom region of the chamber wall 19 is held for movement on the storage chamber 16 and by way of the latter on the mouthpiece 3. The guiding portion 22, storage chamber 16 and mouthpiece 3 are accordingly to be considered an inseparable unit, which can be moved as a whole along the axis x.

To form a means for preventing twisting of the discharge unit A that is formed by the mouthpiece 3, the storage chamber 16 with the molded-on transfer portion 21 and the guiding portion 22, the guiding portion 22 has radial projections 24 disposed underneath a guiding portion top 23 that is aligned transversely in relation to the axis x. These projections engage in correspondingly positioned guiding grooves 25 in the housing wall 5 that run parallel to the axis x-x.

The chamber base 20 is covered over on the chamber side, i.e. facing the substance 17 that is accommodated, by a sliding insert 26 of a form made to match, correspondingly likewise funnel-shaped. This is secured with positive engagement in the region of the chamber base 20 and further extends in coaxial alignment in relation to the axis x into the transfer portion 21, doing so with an inside diameter that is reduced with respect to the transfer portion 21. This region of the sliding insert 26, which is in the form of a cylindrical portion, at the same time forms a sealing portion 27.

Underneath this sealing portion 27, i.e. remote from the storage chamber 16, the wall of the transfer portion 21 is provided with tangentially aligned branch channels 28. These open out radially outward into an air storing space 29.

In the annular space left between the transfer portion 21 and the guiding portion 22 aligned coaxially with it, there engages a coaxially aligned, rotationally symmetrically formed air plunger 30. Viewed in the axial direction, this is formed with a stepped plunger face, thus initially with a radially outer annular plunger face, which provides a radially outward seal against the inner wall of the guiding portion 22 by way of a sealing lip 31. From this annular plunger portion 32 there extends a coaxially aligned tubular portion 33, enclosing the transfer portion 21, into a region that protrudes beyond the transfer portion 21 and in which the plunger face continues radially inward.

The entire discharge unit A is movable in the axial direction relative to the air plunger 30, as a result of which the volume of the air storing space 29 that is formed between the guiding portion 22, the transfer portion 21 and the air plunger 30 is also variable.

The pot wall 33 of the air plunger 30 is enclosed by a coaxially aligned wall 34, which is connected to the air plunger 30 integrally in the same material. Accommodated in the annular space left between this wall and the pot wall 33 is a compression spring 35, which is supported at one end on a top portion of the air plunger 30, which connects the wall 34 to the pot wall 33, and at the other end on a base portion 36 of the inhaler housing 2.

Radially protruding, diametrically opposed driving lugs 37 protrude from the wall 34 enclosing the compression spring 35. These lugs engage with positive engagement in a fixing tube 38 enclosing the guiding portion 22 and the storage chamber 16 radially on the outside, which fixing tube 38 extends approximately from the housing base 36 to underneath the bottom of the mouthpiece. In the end portion facing the mouthpiece 3, the fixing tube 38 is provided on the outer wall with a peripheral latching groove 39. A latching ring 40, which is spring-loaded radially inward, engages in this latching groove 39 for fixing the air plunger 30 in a latching manner against axial displacement by way of the fixing tube 38.

The latching ring 40 is fixed in the inhaler housing wall 5 and has a substantially annular outline, diametrically opposed portions of the latching ring 40 being exposed in the region of indentations 41 made in the outside of the housing wall to match fingers for pressure to be radially applied. Pressure acting on the latching ring 40 radially inward from both sides, for example by means of an index finger and a thumb, leads to a corresponding radial displacement outward of the latching ring portions disposed offset in plan view by approximately 90° in relation to these pressure-application portions, resulting in release of the fixing tube 38 secured in a latching manner.

Furthermore, extending centrally through the inhaler housing 2, along the axis of rotation x, is a rod-like body 42. This is held at the bottom in the region of the housing base 36 and from here extends initially through the radially inner region of the air plunger 30. A further sealing lip 43, provided in the region where it passes through, butts against the lateral wall of the rod-like body 42 for further sealing of the air storing space 29.

The rod-like body 42 extends further through the transfer portion 21, after that through the storage chamber 16 and finally through the mouthpiece 3 in the region of the discharge opening 14 and forms at the end, i.e. in the region of the free end associated with the mouthpiece 3, a nozzle-like discharge portion 44. Extending centrally through the rod-like body 42 is a branch-channel-like air channel 45, which, in a not-in-use position according to the representation in FIG. 3, extends from the discharge portion 44 into the region of the region of the transfer portion 21 that is shielded by the sealing portion 27. Here, the air channel 45 opens in a T-shaped manner; radial openings 46 extending radially outward form correspondingly. Five such radial openings 46 are provided, disposed uniformly around the axis of the air channel.

Secured in the region of the chamber top 18 through which the rod-like body 42 extends is a double-lipped stripping seal 47, which acts against the outer wall of the rod-like body 42.

Approximately midway along the portion in which it extends within the storage chamber 16—with respect to the not-in-use position represented in FIG. 3—the rod-like body 42 carries a dosing chamber D. This is formed as an annular groove 48, runs in a plane perpendicular to the axis x and is formed on the outer lateral wall of the rod-like body 42. This groove is dimensioned in its radial depth such that a wall of solid material remains between the annular groove 48 and the central air channel.

For charging the dosing chamber D or the annular groove 48, filling means B are provided. For charging, these have scooping portions 49, which are suspended from the underside of the chamber top 18, by means of elastically deflectable and returnable arms 50, in such a way that the filling means B move through the supply of substance in the storage chamber 16. Four such filling means B are provided, placed offset at equal angles in relation to one another around the rod-like body 42.

The end portions of the arms 50 remote from the scooping ends 49 have radially inwardly facing thickened pieces 51, which receive a mounting disk 52 enclosing the rod-like body 42. The mount 52, preferably consisting of an elastic thermoplastic or a rubber material, has a central hole of a diameter that is made to match the outside diameter of the rod-like body 42.

In the initial state, i.e. in the not-ready position, the filling means B lie with the thickened pieces 51 against the underside of the chamber top 18. The mounting disk 52 has assumed a position with respect to the rod-like body 42 between two radial beads 53, which are spaced apart from one another in the axial direction and have been machined from the wall of the rod-like body.

The axial length of the rod-like body 42 is furthermore chosen such that the discharge portion 44 provided at the end extends beyond the free peripheral edge of the mouthpiece 3, protruding directly underneath the top 10 of the closure cap.

The way in which the inhaler 1 functions is as follows:

The inhaler 1 is put into operation by unscrewing the closure cap 4. In the course of this unscrewing action and the accompanying axial displacement of the closure cap 4, the entire discharge unit A is displaced by way of the driver 9 on the cap over the same axial distance along the axis x.

The air plunger 30, acted upon rearwardly by the already biased compression spring 35, remains in its starting position as a result of the latching connection between the latching ring 40 and the fixing tube 38. As a result of the axial displacement of the discharge unit A, the air storing space 29 in front of the air plunger 30 is correspondingly increased in size.

In the course of the axial displacement as a result of the turning of the cap, the storage space 16 moves relative to the dosing chamber D. In a position according to the representation in FIG. 4, in which the funnel-shaped chamber base 20 is disposed at a small distance underneath the dosing chamber D, the scooping action of the dosing chamber D with a reproducible amount of the substance 17 takes place. This takes place by the elastically suspended arms 50 being pivoted radially inward while the substance 17 passed through is transported in a scooping manner by means of the scooping ends 49 in the direction of the annular groove 48 of the dosing chamber.

The radial pivoting displacement of the arms 50 of the filling means B takes place in a positively controlled manner by derivation from the relative displacement between the dosing chamber D and the storage chamber 16. In the course of this relative displacement, the scooping ends 49 butt against the chamber base 20, which falls away in a sloping manner in the form of a funnel, or against the sliding insert 26 provided here, which effects the desired deflection against the returning force of the arms 50.

As a result of this configuration, scooping occurs and the dosing chamber D is filled, both at the lowest point of the storage chamber 16, which makes complete emptying of the storage chamber 16 possible.

The scooping ends 49 meet one another, enclosing the annular groove 48 filled with the substance; they correspondingly form a closed form for the annular groove opening, so that the substance introduced cannot escape. The exact apportioning is ensured as a result.

The filling of the dosing chamber D takes place at a point at which the closure cap 4 is still in screw engagement with the inhaler housing 2.

As a result of the further screw displacement of the closure cap 4 to remove the same, the discharge unit A is correspondingly displaced further axially in the direction in which the cap is pulled off, with further displacement of the mouthpiece 3 out of the position in which it is lowered with respect to the inhaler housing 2 into a position in which it is exposed with respect to it. The relative displacement involved with respect to the inhaler housing 2 and correspondingly with respect to the fixed rod-like body 42 has the effect of bringing the discharge portion 44 on the rod-like body closer to the chamber top 18 delimiting the discharge opening 14 at the base of the mouthpiece 3, and consequently into the effective region of the lateral mouthpiece air inlets 15. Accompanying this, the dosing chamber D, which initially is still closed by the scooping ends 49 of the filling means B, leaves the storage chamber 16 and enters the diameter-matched region of the sealing portion 27. This achieves transfer of the filled dosing chamber without any loss.

The scooping ends 49 of the filling means that are initially also supported on the chamber base 20 in the course of this further axial displacement lead to an entrainment of the filling means B together with the entire discharge unit A, while overrunning the radial bead 53 of the rod-like body 42 that correspondingly is encountered in the displacement path, which is made possible by the elastic configuration of the mounting disk 52. The elasticity of this mounting disk 52 is moreover set such that adequate force for overcoming the radial bead 53 is only built up as the scooping ends 49 are acted upon in an abutting manner by way of the storage chamber base 20. The forces acting on the scooping ends 49 during the sliding and the radial displacement accompanying the sliding for the scooping action of the dosing chamber D are not adequate to overcome the retaining forces between the mounting disk 52 and the radial bead 53.

After overrunning the corresponding radial bead 53, the filling means B lose their supporting retention, as a result of which the returning forces of the resiliently designed arms 50 come into effect. Sliding over the sliding insert 26, the filling means B return again into their outstretched original position, with at the same time axial displacement of the thickened pieces 51 and the mounting disk 52 carrying them (cf. FIG. 6).

Unscrewing of the closure cap 4 has the effect of reaching a position in which the mouthpiece 3 has moved almost entirely into an exposed position with respect to the inhaler housing 2. This position is stop-limited. The projections 24 protruding radially from the guiding portion 22 engage against a peripheral edge formed at the ends of the guiding grooves 25 accommodating them.

In this position, the dosing chamber D has reached the transfer point U. In this position, the dosing chamber D is positioned at the level of the branch channels 28 of the transfer portion; thus it is correspondingly brought to the air flow path.

By unscrewing the closure cap 4, the inhaler 1 is automatically brought into the ready position. After that, all that is necessary for the apportioned substance to be expelled under the effect of compressed air from the discharge portion 44 is the triggering of the compression spring 35.

This triggering can be performed by the user himself, as a result of producing a suction pressure. The embodiment represented is one in which the triggering of the compression spring 35 takes place by actuation of the latching ring 40, which inhibits axial movement of the fixing tube 38, by pressure being applied according to the arrows P that are represented in FIG. 7. This has the effect that the latching ring 40 leaves the positive connection with respect to the fixing tube 38, as a result of which the biasing of the compression spring 35 is abruptly ended, with corresponding axial displacement of the air plunger 30. Accordingly, the air in the air storing space 29, which has increased in size by a multiple as the cap is unscrewed, is forced out by way of the tangentially aligned branch channels 28 of the transfer portion 21, which effects a clearance of the dosing chamber D with positive influencing by the circular flow induced by the tangential alignment of the branch channels. The air/substance mixture flows through an annular space 54, left between the rod-like body 42 and the inner wall of the transfer portion 21, in the direction of the free end of the transfer portion 21, in order to enter the air channel 45 here through the transversely aligned radial openings 46.

The annular space 54 is closed at the bottom toward the end of the transfer portion by means of a seal 55.

The substance forced out into the discharge opening 14 by way of the discharge portion 44 is inhaled, breathing in simultaneously occurring and a stream of suction air being built up thereby.

The axial displacement of the air plunger 30 is stop-limited. In this position, the annular, radially outer plunger face butts against the underside of the guiding portion top.

After inhalation is complete, screwing the closure cap 4 back on has the effect that the inhaler 1 is brought into the initial state according to FIG. 3, while taking along the entire discharge unit A, so that the mouthpiece 3 is moved back again into the originally lowered position. In the course of this return displacement of the discharge unit A, the fixing tube 38 is also forcibly displaced back by way of the bottom radial collar 13 of the mouthpiece 3 and the air plunger 30 is forcibly displaced back by way of said fixing tube, until they are in a position in which the latching ring 40 under spring loading falls into the latching groove 39 of the fixing tube 38, for the fixing of the air plunger 30 in a latching manner under the applied spring biasing.

Also in the course of the return displacement of the discharge unit A, the dosing chamber D is brought again into the region of the storage chamber 16. By being entrained in a positively controlled manner, the filling means B assume their starting position, in which their mounting disk 52 again engages in the portion between the two radial beads 53.

The inhaler 1 represented also has a counting device Z. This is provided in the bottom region of the inhaler housing 2 and has firstly a toothed ring 56, which is aligned coaxially in relation to the axis x and has a scale 57, which is provided on its outside and can be read off through a window 58 formed in the housing 2.

The toothed ring 56 cooperates with a gear mechanism 59, which comprises two gear wheels 60 and 61 disposed in a rotationally fixed manner in relation to each other, a smaller gear wheel 60 cooperating with the toothed ring 56.

The larger gear wheel 61 is acted upon for every inhaling action, this effect being derived from the necessary axial displacement of the discharge unit A relative to the housing 2. Thus there protrudes downward from the associated radial projection 24 a switching finger 62, which acts on the gear wheel 61 after inhalation and after the closing screwing-on of the closure cap 4 and the accompanying return displacement of the discharge unit A.

Such a counter is known for example from DE 102004042837, which is not a prior publication. With regard to the mechanics and operating mode of this counter, reference is made in full to the content of this patent application, including for the purposes of incorporating features of this application in claims of the present invention.

All disclosed features are (in themselves) pertinent to the invention. The disclosure content of the associated/accompanying priority documents (copy of the prior application) is also hereby incorporated in full in the disclosure of the application, including for the purpose of incorporating features of these documents in claims of the present application.

1: Inhaler (1) for powdery, especially medical substances (17), with an air channel (45) leading to a mouthpiece (3), a storage chamber (16) for the substance (17) and a moved dosing chamber (D) for apportioning a specific amount of substance from the storage chamber (16) into the region of a transfer point (U) to the air stream, a filling means (B) that charges the dosing chamber (D) in a scoop-like manner being moved right through the supply of substance to the dosing chamber (D), wherein the scoop-like charging by the filling means (B) is derived from a straight relative movement along the longitudinal axis of the inhaler between the dosing chamber (D) and the storage chamber (16).

2: Inhaler according to claim 1, wherein the relative movements of the dosing chamber (D) on the one hand and of the filling means (B) on the other hand take place substantially in directions perpendicular to one another relative to the storage chamber (16).

3: Inhaler according to claim 1, wherein the dosing chamber (D) is formed in a rod-like body (42) extending through the storage chamber (16).

4: Inhaler according to claim 1, wherein the rod-like body (42) at the same time forms a conduit portion.

5: Inhaler according to claim 1, wherein the filling means (B) is connected to a body forming the dosing chamber (D).

6: Inhaler according to claim 1, wherein the movement of the filling means (B) toward the dosing chamber (D) takes place as a result of a sloping positioning of the storage chamber wall (19) with respect to the direction of movement of the dosing chamber (D).

7: Inhaler according to claim 1, wherein the dosing chamber (D) is formed as an annular groove (48) on the outer side of the wall of the rod-like body (42).

8: Inhaler according to claim 1, wherein four filling means (B) are provided, distributed at equal angles around the annular dosing chamber.

9: Inhaler according to claim 1, wherein with the completion of the filling of the dosing chamber (D), the latching connection of the filling means (B) to the dosing chamber body is overcome.

10: Inhaler (1) for powdery, especially medical substances (17), with an air channel (45) leading to a mouthpiece (3), a storage chamber (16) for the substance (17) and an at least linearly moved dosing chamber (D) for apportioning a specific amount of substance from the storage chamber (16) and bringing the amount of substance into a transfer position (U), for transfer to the air stream, it being possible furthermore by relieving a compression spring (35) to produce a stream of compressed air for blowing the substance (17) out of the dosing chamber (D) independently of aspiration, wherein the inhaler (1) can be closed by a closure cap (4) and in that the compression spring (35) is tensioned as the closure cap (4) is placed onto the inhaler (1).

11: Inhaler according to claim 10, wherein the dosing chamber (D) is moved into the filling starting position as the closure cap (4) is placed onto the inhaler (1).

12: Inhaler according to claim 11, wherein the dosing chamber (D) is moved toward an air storing space (29), which can be acted upon by means of the compression spring (35), as the closure cap (4) is removed.

13: Inhaler according to claim 11, wherein the air storing space (29) forms between an air plunger (30), acted upon by the compression spring (35), and a plunger receiving wall as the closure cap (4) is removed.

14: Inhaler according to claim 11, wherein the dosing chamber (D) is disposed such that it radially coincides with the air storing space (29) before the compression spring (35) is triggered.

15: Inhaler according to claim 11, wherein the dosing chamber (D) communicates with a conduit portion, through which the air/substance mixture can be discharged, the compressed air being able to flow into the conduit, clearing the dosing chamber (D), through a radial opening (46) disposed at a distance from the dosing chamber (D).

16: Inhaler according to claim 11, wherein the compressed air flows to the dosing chamber (D) in tangential alignment.

17: Inhaler according to claim 11, wherein the dosing chamber (D) and the conduit are disposed in a central rod-like body (42) that is fixed relative to the inhaler housing (2).

18: Inhaler according to claim 11, wherein the release of the compression spring (35) takes place by actuating a triggering button.

19: Inhaler according to claim 11, wherein the release of the compression spring (35) is effected by the user, as a result of developing a suction pressure.
